(19) United States
(12) Patent Application Publication
(10) Pub. No.: US 2022/0362428 A1
(43) Pub. Date: Nov. 17, 2022

(54) BIOLOGICAL INDICATOR CRUSHING, TYPE-MODEL DETECTION SYSTEM AND METHOD

(71) Applicant: Cem ÖZDAMAR, Izmir (TR)

(72) Inventor: Cem ÖZDAMAR, Izmir (TR)

(21) Appl. No.: 17/625,909

(22) PCT Filed: Apr. 7, 2021
(86) PCT No.: PCT/TR2021/050324

§ 371 Date: Jan. 10, 2022

(30) Foreign Application Priority Data
Oct. 9, 2020 (TR) 2020/16115

(51) Int. Cl. A61L 2/28; C12Q 1/22; G01N 31/22

(52) U.S. Cl. CPC A61L 2/28; C12Q 1/22; G01N 31/22

(57) ABSTRACT

A system that enables automatic biological indicator crushing and determination of biological indicator type and model in incubator devices with biological indicator readers.

### TECHNICAL AREA

The present invention relates to a system and method that automatically crushes the ampoule in the structure of rapid type biological indicator products used for the control of the sterilization process in the incubator device with an automatic reader and enables the incubator device to automatically determine the incubation program by detecting the biological indicator cap color.

### KNOWN STATE OF THE TECHNIQUE

The sterilization biological indicators contain a paper strip/disc impregnated with bacteria spores resistant to the sterilization process, enclosed in the transparent plastic tube (B) shown in FIG. 1 and a culture medium (C) filled in a glass ampoule that enables the germination of spores. After the sterilization process, as seen in FIG. 1, the plastic tube (B) is pressed from the outside with a finger or by using various apparatus; the glass ampoule is broken manually and contacted with the spores. If the spores are alive after the sterilization process, the process is unsuccessful; the spores in the medium will germinate (positive result). Germinated spores either cause a color change in the PH indicator added to the nutrient or cause fluorescence emission by the enzymes they produce so they can be detected earlier by the reader incubators in rapid types . . . .

In the current technique/practice, three methods are used to crush the ampoule in the biological indicator as mentioned in patents U.S. Pat. No. 8,173,388, WO2009/151886, US20170253905A1, CN103343091:
Crushing by hand. To do this, the thumb and forefinger are pressed either in the middle of the tube in most models or on the cap of the tube in some models.
Crushing with the ampoule crushing well in the incubator structure.
Crushing with an ampoule crusher.

In current applications, the methods mentioned above require the use of manual force and could potentially cause hurt/injury and/or loss of time and labor. Therefore, the need for an automatic biological indicator crushing apparatus has been identified. This automated system eliminates the above drawbacks and disadvantages in rapid biological indicator automatic reader incubator devices, and reduces the risk of human error.

There are various types of biological indicators. For example, common with sterilization methods are Steam, Hydrogen Peroxide and Ethylene Oxide. Formaldehyde can also be used. In addition, there are different models in rapid types, each type, mainly instant, 1 hour for 20 minutes or 4 hours . . . .

In order to determine whether processed rapid biological indicators will give positive or negative results, first the type and model must be introduced to the automated system reader. The program to be applied by the reader is to each type and model. The generally applied method is to manually adjust the parameters from the reader interface as seen in FIG. 6 and/or to add additional barcode or electronic label (rfid) alike systems on the biological indicator, as seen in FIG. 7, to be recognized by the reader in a seperate process step, before starting the incubation, it allows the reader to be detected manually. Ultimately, the reader recognises the biological indicator and decides which operating program to implement. However, these applications have two disadvantages. These are as follows:
Faulty operation and result if the the operator chooses incorrect parameters
Electronic label/barcode etc., which must be added as extra on the biological indicator, and labour and time wasted during the promotion process.

However, biological indicators are already produced in different plastic cap (A) colors according to their traditional structures, types and models. For example;
Steam Type 180 minutes model: Brown
Steam Type 60 minutes model: Magenta
Steam Type 20 minute model: Light Blue
Steam Type Instant model: Black
Ethylene Oxide Type 240 minute model: Green
Hydrogen Peroxide Type 60 minute model: Grey
Hydrogen Peroxide Type 20-minute model: White
Formaldehyde Type 60 minute model: Red
Within the framework of these distinctions, which have become almost standard day by day, different manufacturers are trying to meet on a common platform for common identification.

By using the cap color specific to the biological indicator type and model as a distinguishing feature, the reader can automatically determine the operating program with no extra processing or user intervention. For this purpose, it suffices to have a built-in color identification unit for the biological indicator in the reader structure. Even assuming that there is no common color application between different manufacturers for type and model each manufacturer can create a solution by matching his reader and biological indicator.

Because of the research on the subject, the application numbered CN103343091 was found. In the system, for hydrogen peroxide sterilizers, it is mentioned about crushing the biological indicator ampoule manually inserted by the operator with a clamp in the incubator structure integrated into the body; it does not have an automatic feature.

As a result, it was required to improve in the relevant technical field because of the above negativities and the insufficiency of existing solutions.

### PURPOSE OF THE INVENTION

The invention is created by being inspired by the current situations and aims to solve the problems mentioned above.

The primary purpose of the invention is to develop an automatic biological indicator crushing device and a cap color recognition system for automatic biological indicator type and model identification for rapid biological indicator incubators with an automatic reader.

Another aim of the invention is to prevent possible injuries by ensuring that biological indicators are broken without touching and to raise security to higher levels.

Another aim of the invention is to prevent loss of labor and time for crushing biological indicators.

Another aim of the invention is to automatically determine the type and model of biological indicators used for testing and to prevent erroneous results that may arise because of faulty working program and parameter selection.

Another aim of the invention is to eliminate the requirement of electronic tags/barcodes, etc. used on the biological indicator in order to determine the biological indicator type and model.

Another aim of the invention is to ensure that the system benefits by increasing security, facilitation of use and achieving safer results and increasing the use of rapid biological indicators

Invention to fulfill the above objectives is the system that allows automatic biological indicator crushing and biological indicator type and model determination in incubator devices with indicator reader. The system;
the biological indicator well(s) in the reading log, which is part of the biological incubator device, allowing the biological indicators to crush and
   a motor that provides the movement for the crushing process to take place,
   a mini motor with recution gear
   a drive flywheel that is connected to the reduction gear output mentioned and rotates circularly,
   a moving skid that moves back and forth, approaching and moving away from the indicators,
   the fixed skid that allows it to move on the mentioned moving skid,
   the crushing pins mounted on the lying slid mentioned, which allow the glass ampoule inside the biological indicator to crush,
   a sensor that detects the position of the mentioned moving skid and informs the biological indicator reader
crushing apparatus containing,
can detect the colors of the caps of biological indicators in the biological indicator well(s) in the reading log, which is part of the biological incubator device, on the aforementioned crushing apparatus; and
   at least one sensor capable of detecting the color of biological indicator caps with detection beams sent through the detection hole created on the biological indicator reader cap,
   electronic board that provides data from the sensor with coupled with the mentioned sensor to the biological indicator reader
including color recognition system

The invention also caps the method that allows automatic biological indicator crushing and biological indicator type and model determination in incubator devices with indicator reader. The method accordingly;
crushing the crushing apparatus by:
   circular rotation of the drive flywheel connected to the output of the motor coupled reductor, which provides the movement for the realization of the crushing process, and the moving skid moving on a fixed skid moves back and forth, approaching and moving away from the biological indicator,
   a position sensor to report the status of the moving skid to the rapid biological indicator reader device, which is part of the incubator device,
   crushing pins mounted on the mentioned moving slid crushing the glass ampoule inside the biological indicator during the approach.
the color recognition system works:
   detection beams sent through the detection hole created on the indicator reader cap and biological indicator cap color detection of the sensor that can detect the color of the biological indicator caps,
   sending biological indicator cap color data to rapid biological indicator reader device with an electronic board,
   incubator device with rapid biological indicator reader automatically determines the biological indicator model in line with the data it receives from sensors
      it contains the process steps.

The structure and characteristics of the invention and all its advantages are clearly understood by the figures below and the detailed explanation given in these figures, and therefore the assessment must be made in consideration of these figures and detailed explanation.

### FIGURES TO HELP UNDERSTAND THE INVENTION

FIG. 1 shows biological indicators used in the current technique and the broken image of the ampoule inside these biological indicators.

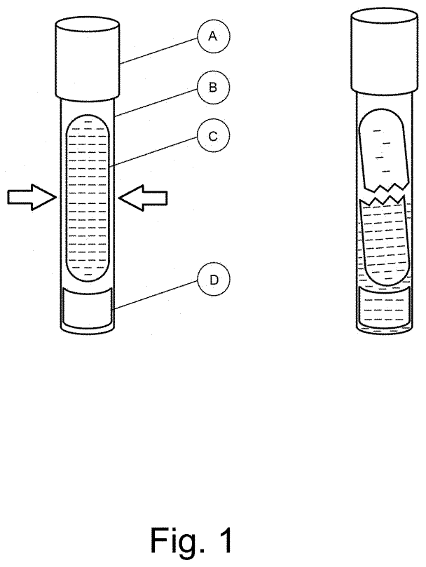

FIG. 2 provides drawings showing the method of crushing with the help of the hand used in the current technique to crush the ampoule inside the biological indicator.

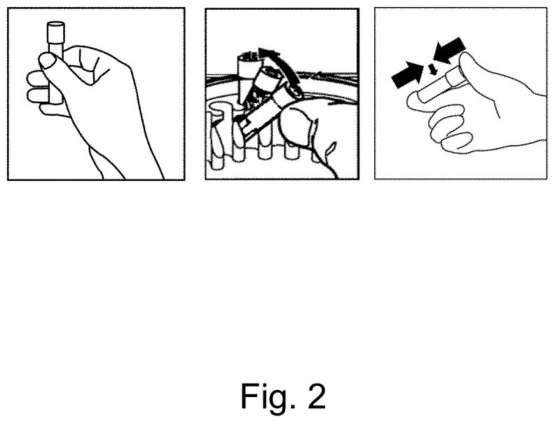

FIG. 3 gives the drawings showing the method of crushing the ampoule in the current technique to crush the ampoule in the biological indicator.

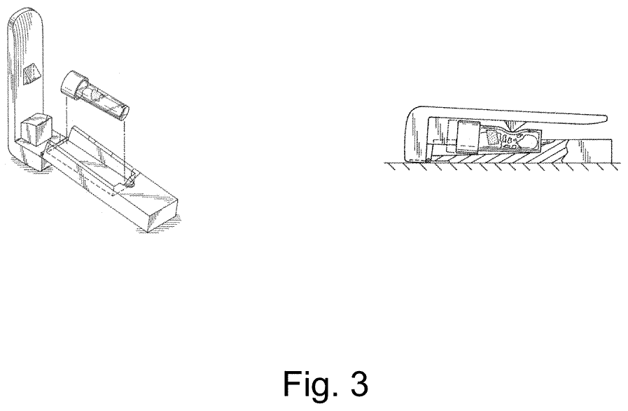

FIG. 4 gives the drawings showing the ampoule crushing well and method used in the current technique to crush the ampoule in the biological indicator.

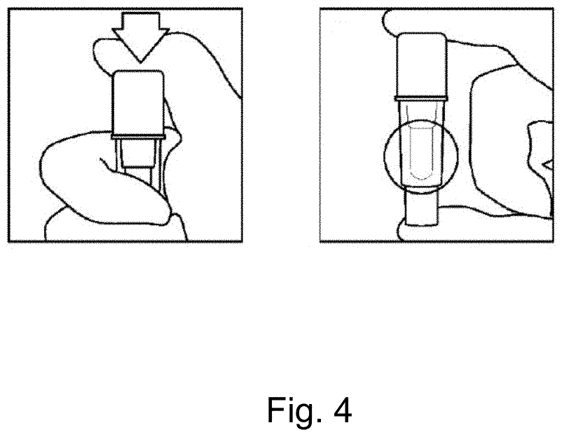

FIG. 5 gives the drawings showing the manual parameter setting application from the reader interface.

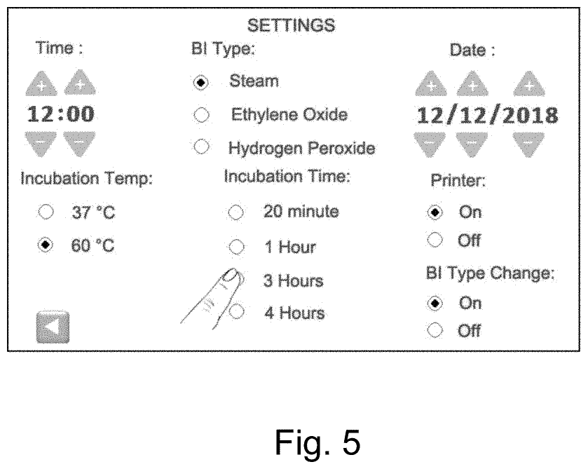

FIG. 6 gives drawings showing applications in barcode or electronic label type on the biological indicator in a way that the reader can recognize.

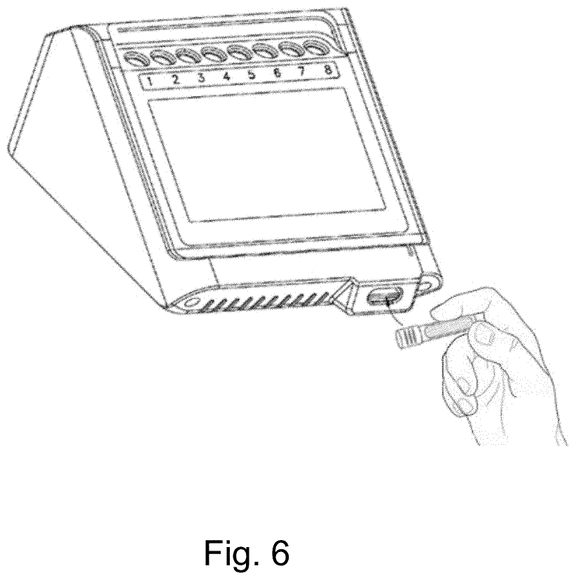

FIG. 7 displays the automatic crushing apparatus in the system subject to the invention.

FIG. 8 gives the image of the color recognition system in the system subject to the invention.

Description of Part References

1. Reducer
2. Motor
3. Moving skid
4. Indicator well
5. Reading log
6. Biological Indicator
7. Crushing pad
8. Drive flywheel drive
9. Position sensor
10. Fixed skid
11. Detection beam
12. Biological Indicator cap
13. Detection hole
14. Sensor
15. Electronic board
A: Plastic cap
B: Plastic Tube
A: Cultural environment
D: Bacteria spores impregnated on
paper

### DETAILED DESCRIPTION OF THE INVENTION

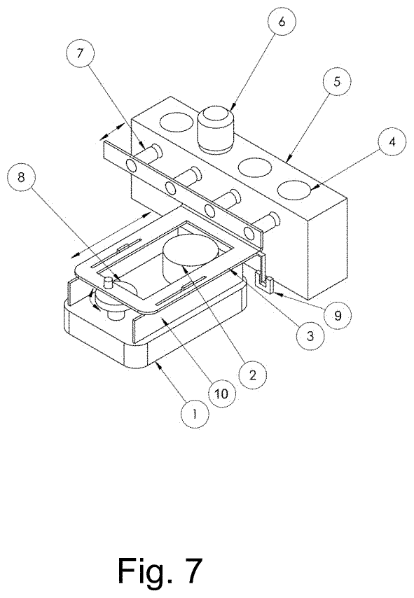

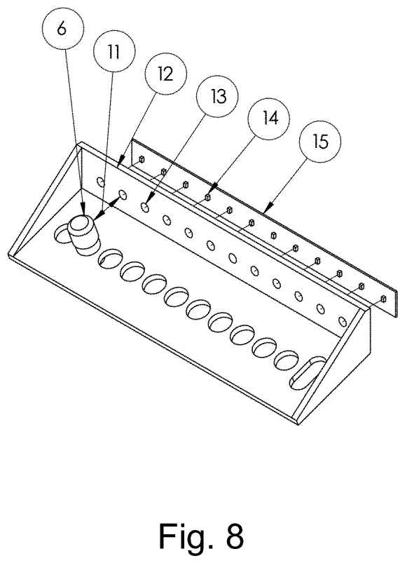

In this detailed explanation, the preferred configurations of the system subject to the invention are explained only for a better understanding of the subject. The invention is the system that allows automatic biological indicator (6) crushing and biological indicator (6) type and model determination in incubator devices with biological indicator reader. In FIG. 7 and FIG. 8, provide images of the system subject to the invention are given. The system; allows the indicators (6) in the indicator well (4) in the reading log (5), which is part of the incubator device, to and the motor (2), which provides the movement for the crushing process to take place, reducer (1) join to the motor mentioned (2) the drive flywheel (8), which is connected to the output of the mentioned reductor (1) and rotates circularly, moving slide (3) moving back and forth moving closer to and away from the indicators (6) fixed skid (10), which allows the mentioned moving skid (3) to move on it, mounted on the mentioned moving sling (3) and crushing pins (7) that allow the glass ampoule inside the biological indicator (6) to crush, crushing apparatus containing sensor (9) that detects the position of the mentioned moving skid (3) and informs the biological indicator reader, on the aforementioned crushing apparatus, at least one sensor (14) that can detect the colors of the caps of the indicators (6) in the reading log (5), which is part of the incubator device, and detect the color of the indicator (6) caps with the sensing rays (11) sent through the detection hole (13) created on the indicator reader cap (12), It comprises a color recognition system comprising an electronic board (15) that transmits the data coming from the sensor (14) coupled with the mentioned sensor (14) to the indicator reader.

The motor mentioned in the preferred application of the system (2) is characterised as a DC motor. In another preferred application of the system, the system includes a lighting system that allows the color recognition system to work in low-light environments.

In FIG. 7, an overview of the automatic biological indicator crushing apparatus is given. Biological indicators (6) are placed in the indicator well (4), which is in the reading log (5), which is part of the incubator device with a rapid biological indicator reader through which the reading is passed. The reading log (5) is also heated to a certain temperature according to the type of biological indicator (6) and its temperature is maintained. The crushing apparatus is a unit independent of the reading log but preferably placed next to the reading log.

The process of crushing the method subject to the invention is:

The crushing apparatus comprises motor (2), reduction gear unit (1), drive flywheel (8), fixed skid (10) and moving skid (3) and moving skid-mounted crushing pins (7). The drive flywheel (8) connected to the reducer(1) output turns circular, but the moving skid (3) moving on the fixed skid (10) is linear. Moving back and forth, the moving skid (3) approaches the biological indicator (6) and moves away. Crushing pins (7) mounted on the moving sling (3) quickly crush the glass ampoule inside the biological indicator (6) during the approach. The position sensor (9) informs the state of the moving skid (3) to the rapid biological indicator reader device, which is part of the incubator device. The rapid biological indicator reader device automatically crushes the glass ampoule inside the biological indicator (6) by using and controlling the crushing apparatus with the data it receives from the position sensor (9).

The crushing apparatus is usually positioned under the rapid biological indicator cap (12). The color recognition system is located just above the crushing apparatus.

The method subject to the invention is color recognition:

FIG. 8 summarizes the color recognition system. The system comprises sensors (14) and an electronic board (15) to which it is coupled. The biological indicator has a sensing hole (13) on it to detect the cap color. Biological indicators placed in the biological indicator well (4) automatically detect (6) through cap-color sensors (14) and send biological indicator (6) model data to the electronic board (15) and a rapid biological indicator reader device. The incubator device with rapid biological indicator reader can automatically determine the biological indicator (6) model in line with the data it receives from sensors (14).

1. A biological indicator system that enables automatic biological indicator crushing and biological indicator type and model identification in incubators with readers, wherein:
the biological indicators (6) in the biological indicator well(s) in the reading log which is part of the biological incubator device, and
a motor that-provides the movement for the crushing process to take place;
a reducer joins to the motor,
a rotating drive flywheel is connected to an output of the reducer or reduction (1) and rotates circularly,
a moving slide moving back and forth, moving closer to and away from the biological indicators;
a fixed skid allowing the moving skid to move on,
crushing pins mounted on the mentioned moving slid and allowing the glass ampoule inside the biological indicator to crush,
a sensor that detects the position of the mentioned moving skid and informs the biological indicator reader,
containing crushing apparatus,
can detect the colors of the caps of biological indicators in the biological indicator well in the reading log, which is part of the incubator device, on the afore mentioned crushing apparatus, and
at least one sensor, which can detect the color of the indicator caps with detection rays sent through a detection hole, formed on the indicator reader cap,
an electronic board that transmits the data coming from the sensor coupled with the mentioned sensor to the indicator reader Including color recognition system.

2. The system of claim 1, wherein the color recognition system includes a lighting system that allows it to work in low light/dark environments.

3. The system of claim 1, wherein the motor is a DC motor.

4. The system of claim 1, comprising positioning of the afore mentioned crushing apparatus under the biological indicator reader cap.

5. The system of claim 1, comprising positioning the mentioned color recognition system on the crushing apparatus.

6. A a method that provides automatic biological indicator crushing and biological indicator type and model determination in incubators with biological indicator readers, the method comprising:
performing the crushing process of the crushing apparatus:
with the circular rotation of the drive flywheel connected to the output of the coupled reductor to the motor, which provides the movement for the crushing process, and the moving skid moving on a fixed skid moves back and forth to the biological indicator,
a position sensor notifying the state of the moving skid to the rapid biological indicator reader device, which is a part of the incubator device,
crushing the glass ampoule inside the biological indicator during the approach of the crushing pins mounted on the mentioned movable slide,
the color recognition system works:
detection beams sent through the detection hole created on the indicator reader cap and the biological indicator of the sensor that can detect the color of the caps of the sensor biological indicator,
sending biological indicator cap color data to rapid biological indicator reader device with electronic board,
automatic determination of the biological indicator model in line with the data received from the sensors by the incubator device with rapid biological indicator reader.
